(19) United States
(12) Patent Application Publication
(10) Pub. No.: US 2024/0075002 A1
(43) Pub. Date: Mar. 7, 2024

(54) CREATINE COMPOSITION AND METHODS OF USING SAME

(71) Applicant: NanoChem Solutions, Inc, Peru, IL (US)

(72) Inventors: GEORGE MURRAY, Peru, IL (US); Daniel O'Brien, Peru, IL (US); Keith Schmidt, Peru, IL (US)

(73) Assignee: NanoChem Solutions, Inc, Peru, IL (US)

(21) Appl. No.: 18/460,144

(22) Filed: Sep. 1, 2023

Related U.S. Application Data
(60) Provisional application No. 63/402,990, filed on Sep. 1, 2022.

(51) Int. Cl. A61K 31/198; A61K 9/16

(52) U.S. Cl. CPC A61K 31/198; A61K 9/1611; A61K 9/1688

(57) ABSTRACT

A creatine composition comprising a base component, a dianion component, and a creatine component. In some exemplary embodiment, the base component and dianion component can at least be partially reacted before being added to the creating component. On a dry weight basis, the base component can comprise between about 7-20% of the composition by weight, the dianion component can comprise between about 35-50% of the composition by weight, and the creatine component can comprise between about 30-60% of the composition by weight. In embodiments where the base component and dianion component are first reacted to form a base dianion component, the base dianion component can comprise between about 40-65% of the composition by weight. The creatine component can comprise between about 35-60% of the composition by weight with the remainder of the composition being composed of deionized water (i.e., between about 0-10% of the composition by weight).

This U.S. Patent Application claims priority to U.S. Provisional Application 63/402,990 filed Sep. 1, 2022, the disclosure of which is considered part of the disclosure of this application and is hereby incorporated by reference in its entirety.

### FIELD OF THE INVENTION

This invention relates generally to a dried powder composition comprising a reaction of creatine with an inorganic or organic dianion along with a base. The resulting creatine base dianion compound has greatly increased solubility in aqueous solutions (and therefore bioavailability). Unlike other creatine-acid compounds and admixtures, it can more easily be spray dried, allowing for a production of a pure creatine compound with low moisture content that can be incorporated into a variety of supplements, sports drinks, and foodstuffs.

### BACKGROUND

As disclosed in U.S. Pat. No. 5,973,005, “the energy which enables the muscles in mammals to contract and expand is produced from adenosine triphosphate (ATP). The adenosine triphosphate metabolizes in the muscle by cleaving a phosphate radical to release the energy to contract the muscle and to produce adenosine diphosphate (ADP) as a byproduct. As the amount of adenosine triphosphate is depleted during extended exercise, muscle strength decreases and muscle fatigue increases.

ATP can be produced in the muscle from glycogen or creatine phosphate. Creatine phosphate provides a ready source of phosphate and is able to resynthesize ATP at nearly twice the rate compared to glycogen. The amount of creatine phosphate in the muscle and in the bloodstream are important in the time required to recover from muscle fatigue.

Creatine is produced naturally in humans and other animals and is converted to create phosphate in the muscles. The creatine phosphate is stored in the muscle as an available source of phosphate for the resynthesis of ATP from adenosine diphosphate (ADP).

Creatine is produced in the liver, kidney, and pancreas and is supplied to the body by the food intake. Creatine is only sparingly soluble in water and is normally present in the bloodstream at a concentration of about 50 micromol per liter of blood. The creatine enters the muscle tissue by active transport where it is converted to creatine phosphate. Muscle fatigue and the accumulation of lactic acid occur when the supply of creatine phosphate is exhausted and the ADP cannot be converted to ATP.”

There have been a plethora of creatine salts and admixtures created to in an effort to increase the solubility and therefore the bioavailability of the creatine. Creatine stability is a concern—especially in the acidic environment of the stomach. Sources state that creatine can convert into creatinine—a creatine metabolite that's excreted from the body and does not contribute to the production of ATP. From a formulation perspective, having a compound that can be easily spray dried with high yields is also desirable due to the flexibility in use in formulations as a powder (versus being available only as an aqueous solution). Creatine nitrate and creatine citrate (both without a base counterpart in the compound)—two common creatine admixtures in the industry—do not spray dry well, owing to the hygroscopic nature of both citric acid and nitric acid. Traditional creatine compounds can also have a finished pH that is too low and could be harmful if ingested unbuffered.

Accordingly, a continuing need exists in the industry for an improved creatine compound that is highly soluble, highly bioavailable, and is stable in the low pH of the stomach.

### BRIEF SUMMARY OF THE INVENTION

In one aspect, this disclosure is related to a method of supplying creatine to an animal, and particularly humans, in a soluble, stable form that can be utilized and absorbed by the body. More particularly, the invention is directed to a creatine solution that is dried and to a process producing a stable form of creatine base dianion as the starting material.

In another aspect, the present disclosure is related to a creating composition having a base dianion component and a creatine component. A base component and dianion component can first be partially reacted to form a base dianion component before being reacted with the creatine component. The base component can be any suitable base, including but not limited to sodium, potassium, ammonium, L-lysine, or L-arginine, and ethanolamine. The dianion component can include but is not limited to sulfuric acid, phosphoric acid, L-aspartic acid, L-glutamic acid, malonic acid, succinic acid, fumaric acid, or glutaric acid. The base dianion component formed can include but is not limited to sodium hydrogen sulfate or potassium hydrogen sulfate. The base component can comprises between about 7-20% of the composition by weight, the dianion component can comprises between about 35-50% of the composition by weight, and the creatine component can comprise between about 30-60% of the composition by weight.

In another aspect, this disclosure is related to a method and composition to increase the solubility of creatine. A base component and dianion component can be combined to form a base-dianion component. The base-dianion component can be placed into a solvent to form a base-dianion solution. The base-dianion solution can be heated to at least 140° F. A creatine component can be added to the base-dianion solution to form a creatine solution. The creatine solution can be kept at a temperature of at least 140° F. for a duration of between about 30-90 minutes and dried to a solid formulation having a solvent content between about 0-10% by weight.

In yet another aspect, this disclosure is related to method and composition to spray dry more efficiently.

The invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### DETAILED DESCRIPTION OF THE INVENTION

The following detailed description includes references to the accompanying drawings, which forms a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense.

Before the present invention of this disclosure is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the disclosure made herein.

Unless otherwise indicated, the words and phrases presented in this document have their ordinary meanings to one of skill in the art. Such ordinary meanings can be obtained by reference to their use in the art and by reference to general and scientific dictionaries.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The present disclosure relates to a composition and process of producing a stable solution of creatine base dianion compound. In some exemplary embodiments, the composition of the present disclosure can include a base component, a dianion component, a creatine component, and a solvent component. A base component can function as a counter ion to the dianion, an unexpected stability is added to the creating composition allowing for the composition to be spray dried much more easily that traditional creatine compounds.

In some exemplary embodiments, various inputs can be used to create the creatine base and dianion and can include one or more of the following components. A base component can include but is not limited to one or more of a sodium, potassium, ammonium, L-lysine, L-arginine, and ethanolamine. A dianion component can include but is not limited to one or more of sulfuric acid, phosphoric acid, L-aspartic acid, L-glutamic acid, malonic acid, succinic acid, fumaric acid, or glutaric acid.

In some exemplary embodiments, a creatine component can include any suitable creatine, including but not limited to, creatine monohydrate, creatine anhydrous, creatine ethyl ester, and liquid creatine. Preference can be given to materials with (1) beneficial nutritional properties, (2) low toxicities in the human body, (3) low molecular weights in order to maximize the creatine content as a percent of the finished compound.

The creatine base dianion compound of the present disclosure can be dried into a stable powder. Drying can include any suitable methods of removing water from an aqueous solution such as but not limited to spray drying, drum drying, freeze drying and vacuum drying including combinations of drying processes. The solution can be obtained by reacting a food-grade dianion acid component (or organic or inorganic) with a 1:1 molar ratio of base component. The reaction can then be completed by reacting with a creating component to form an exemplary embodiment of a complete creatine base dianion composition of the present disclosure. In some exemplary embodiments, the composition may have a between 1:1 to about a 1:3 ration of base component to dianion component.

The solubility of creatine in an aqueous solution at room temperature is 1.33 g/100 mL. By creating an exemplary embodiment of a creatine base dianion composition of the present disclosure, such as creatine sodium sulfate or a creatine potassium sulfate ionic salt, the solubility of creatine can be greatly increased and enters into solutions very easily. In some exemplary embodiment, the creatine base dianion composition of the present disclosure can have a pH between about 1.5 and 2.5. By weight, compositions containing 8 g/100 mL of creatine were observed to be very stable at room temperature and remains in solution for a longer duration than current creatine solutions.

Creatine monohydrate is typically converted to the downstream metabolite, creatinine. In some exemplary embodiments of the present disclosure, reacting creatine with a dianion component that has been partially reacted with a base component and supplying heat at a temperature greater than about 140° F., a creatine base dianion anhydrous salt composition can be created, which has a greater stability at lower pH's. Spray-drying the compound can further ensure the composition of the present disclosures stability over a period of time, preventing any possible conversion from creatine to creatinine. In some exemplary embodiments, the heat can be applied between about 140° F. to about 170° F.

In some exemplary embodiments, a creatine composition can include a base component, a dianion component, and a creatine component. In some exemplary embodiment, the base component and dianion component can at least be partially reacted before being added to the creating component. On a dry weight basis, the base component can comprise between about 7-20% of the composition by weight, the dianion component can comprise between about 35-50% of the composition by weight, and the creatine component can comprise between about 30-60% of the composition by weight. In embodiments where the base component and dianion component are first reacted to form a base dianion component, the base dianion component can comprise between about 40-65% of the composition by weight. The creatine component can comprise between about 35-60% of the composition by weight with the remainder of the composition being composed of deionized water (i.e., between about 0-10% of the composition by weight).

In some exemplary embodiments, a base dianion composition can be added to a solvent solution, including but not limited to deionized water. The ratio between the solvent solution and the base dianion composition can be anywhere from between about 10:1 to 1:1 by weight. The solvent solution with the base dianion composition can be heated to about 140° F. or more. A creatine component can then be added to the solvent solution and the temperature maintained throughout the reaction between the base dianion solvent solution and the creatine component. The ratio between the creatine component and the base dianion can be 1:1 with the total creatine base dianion to solvent ratio being anywhere from 10:1 to 1:1. The mixture can additionally be agitated using any suitable means for between about 30 to 90 minutes. The solution can then be dried using any suitable means including but not limited to a spray drier. The generated creatine composition powder can have moisture content of between about 0-10% by weight and with a water activity of less than 0.4. The pH of the powder can be between about 1-6.

In some exemplary embodiments, the creatine composition of the present disclosure can be formed using a base-cation component, an acid dianion component, a creatine component, and a solvent. The base cation component and acid dianion component can collectively comprise between about 10-40% of the composition by weight. The ratio between the first base cation component and second acid dianion component should be about 1:1. In some other exemplary embodiments, one or more creatine base acid dianion components can be utilized.

In one exemplary embodiment, a creatine composition of the present disclosure can be a creatine sodium sulfate salt composition can include a concentration of a base dianion, such as sodium hydrogen sulfate between about 7-8% by weight, and a creatine monohydrate of between about 8.5-9.5% by weight, allowing for a 1:1 molar ratio of sodium hydrogen sulfate and creatine monohydrate. The balance of the formulation can include deionized water. In another exemplary embodiment of a creatine composition of the present disclosure, can include a creatine potassium sulfate salt, with the concentrate of potassium hydrogen sulfate can be between about 10-11% and the creatine monohydrate can be between about 11-12% by weight, allowing for a 1:1 molar ratio of potassium hydrogen sulfate and creatine monohydrate. The balance of the formulation can be a solvent including but not limited to deionized water.

In one exemplary embodiment, an aqueous solution of creatine sodium sulfate can be produced by dissolving about 72.96 grams of sodium hydrogen sulfate in about 811.86 grams of deionized water. The solution can be heated to about 145° F. and is maintained throughout the reaction. When the temperature of the sodium hydrogen sulfate solution reaches about 145° F., about 90.63 grams of creatine monohydrate can be added to the solution whereby the heat is maintained at about 145° F. and the mixture mixes with standard agitation for approximately 1 hour. After the hour, the solution is immediately piped to the spray dryer whereby it is spray dried into a powder with a total water of 3-7% by weight and with a water activity of less than 0.4. The pH of the spray dried powder is approximately 1.7. The mesh size of the spray-dried, stable, soluble sodium hydrogen sulfate powder is approximately 40-200 mesh, but can be adjusted depending on the requirements of the customer or desired end application.

In another exemplary embodiment, an aqueous solution of creatine potassium sulfate can be produced by dissolving about 109.53 grams of potassium hydrogen sulfate in about 806.68 grams of deionized water. The solution is can be heated to about 145° F. and is maintained throughout the reaction. When the temperature of the sodium hydrogen sulfate solution reaches about 145° F., about 119.05 grams of creatine monohydrate can be added to the solution whereby the heat is maintained at 145° F. and the mixture mixes with standard agitation for approximately 1 hour. After the hour, the solution can be immediately piped to the spray dryer whereby it is spray dried into a powder with a total water of 3-7% by weight and with a water activity of less than 0.4. The pH of the spray dried powder is approximately 1.7. The mesh size of the spray-dried, stable, soluble potassium hydrogen sulfate powder is approximately 40-200, but can be adjusted depending on the requirements of the customer.

In yet another exemplary embodiment, an aqueous solution of half creatine potassium sulfate and half creatine sodium sulfate can be prepared by dissolving about 43.00 grams of sodium hydrogen sulfate and about 47.10 grams of potassium hydrogen sulfate in about 881.43 grams of deionized water. The solution can be heated to about 145° F. and is maintained throughout the reaction. When the temperature of the sodium hydrogen sulfate and potassium hydrogen sulfate solution reaches 145° F., 102.39 grams of creatine monohydrate can be added to the solution whereby the heat is maintained at about 145° F. and the mixture mixes with standard agitation for approximately 1 hour. After the hour, the solution is immediately piped to the spray dryer whereby it is spray dried into a powder with a total water of between about 0-10% by weight and with a water activity of less than 0.4. The pH of the spray dried powder is approximately 1.7. The mesh size of the spray-dried, stable, soluble potassium hydrogen sulfate powder is approximately 40-200, but can be adjusted depending on the requirements of the customer.

While the invention has been described above in terms of specific embodiments, it is to be understood that the invention is not limited to these disclosed embodiments. Upon reading the teachings of this disclosure many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

What is claimed is:

1. A creatine composition comprising:
at least one base component;
at least one dianion component;
a creatine component; and
a solvent component.

2. The composition of claim 2, wherein creatine composition can include at least one of the following: anhydrous creatine or creatine monohydrate

3. The composition of claim 2, wherein the base component can include at least one the following: sodium, potassium, ammonium, L-lysine, or L-arginine, and ethanolamine.

4. The composition of claim 2, wherein the dianion component can include at least one of the following: sulfuric acid, phosphoric acid, L-aspartic acid, L-glutamic acid, malonic acid, succinic acid, fumaric acid, or glutaric acid.

5. The composition of claim 1, wherein the base component comprises between 7-20% of the composition by weight on a dry weight basis,
the dianion component comprises between about 35-50% of the composition by weight on a dry weight basis, and
the creatine component comprises between about 30-60% of the composition by weight on a dry weight basis.

6. The composition of claim 7 wherein the base component and dianion component are first reacted to form a base dianion component, wherein the base dianion component can comprise between about 40-65% of the composition by weight.

7. The composition of claim 8, wherein the solvent component comprises between 0.1-10% by weight.

8. The composition of claim 9, wherein the solvent is deionized water.

9. The composition of claim 8, wherein the creatine composition can include at least one of the following: anhydrous creatine or creatine monohydrate;
the base component can include at least one the following: sodium, potassium, ammonium, L-lysine, or L-arginine, and ethanolamine; and
the dianion component can include at least one of the following: sulfuric acid, phosphoric acid, L-aspartic acid, L-glutamic acid, malonic acid, succinic acid, fumaric acid, or glutaric acid.

10. The composition of claim 9, wherein the base component is sodium, the dianion component is sulfuric acid, and the creating component is creatine monohydrate.

11. The composition of claim 9, wherein the base component is potassium, the dianion component is sulfuric acid, and the creatine component is creatine monohydrate.

12. The composition of claim 9, wherein the composition comprises a first base component, a first dianion component, second base component and a second dianion component.

13. The composition of claim 12, wherein the first base component is sodium, the second base component is potassium, the first dianion component is dianion component is sulfuric acid, and the second dianion component is sulfuric acid.

14. A process of producing a creatine composition comprising:
providing a base-dianion component into a solvent to form a base-dianion solution;
heating the base-dianion solution to at least 140° F.;
adding a creatine component to the base-dianion solution to form a creatine solution;
maintain the creatine solution at a temperature of at least at least 140° F. for a duration of between 30-90 minutes;
drying the creatine solution to a solid formulation having a solvent content between 0-10%.

15. The method of claim 11, wherein drying includes all methods of removing water from an aqueous solution such as but not limited to spray drying, drum drying, freeze drying and vacuum drying including combinations of drying processes.

16. The process of claim 11, wherein the creatine composition is creatine sodium sulfate having a pH between 1.5-2.5.

17. The process of claim 11, wherein the creatine composition is creatine potassium sulfate s having a pH between 1.5-2.5.

18. The process of claim 11, wherein the creatine composition is a mixture of creatine potassium sulfate and creatine sodium sulfate having a pH of between 1.5-2.5.

19. The process of claim 11, wherein the base dianion component is sodium hydrogen sulfate.

20. The process of claim 11, wherein the base dianion component is potassium hydrogen sulfate.
